(19) United States
(12) Patent Application Publication
(10) Pub. No.: US 2009/0312660 A1
(43) Pub. Date: Dec. 17, 2009

(54) Recognition and localisation of pathologic animal and human sounds

(75) Inventors: Marcella Guarino, Milan (IT); Sara Ferrari, Stefano (VA) (IT); Annamaria Costa, Vellezzo Bellini (PV) (IT); Federica Borgonovo, Milano (MI) (IT); Daniel Berckmans, Kessel-Lo (BE); Jean-Marie Aerts, Haasrode (BE); Mitchell Silva, Ixelles (BE); Vasileios Exadaktylos, Thessaloniki (BE); Toon Leroy, Heverlee (BE)

Correspondence Address: BACON & THOMAS, PLLC, 625 SLATERS LANE, FOURTH FLOOR, ALEXANDRIA, VA, 22314-1176, US

(73) Assignees: BIORICS NV, Zwijnaarde (BE); K.U. LEUVEN RESEARCH & DEVELOPMENT, Leuven (BE)

(21) Appl. No.: 12/213,254

(22) Filed: Jun. 17, 2008

(51) Int. Cl. A61B 5/08; G06F 17/00

(52) U.S. Cl. 600/529; 702/188

(57) ABSTRACT

A system and method are described for combining the respiratory status (e.g. amount and type of cough) with the localization of organisms having the respiratory status in real time. The organisms are able to suffer from a respiratory complaint, i.e. they have lungs such as mammals especially farm animals and humans. In particular the present invention is advantageous for animals and humans who are exposed to closed confinements such as pens, cages, aircraft, public places where humans are in close proximity to each other.

The present invention relates to system and methods for the detection of pathologic states in mammals.

### TECHNICAL BACKGROUND

Airborne virus and bacterial diseases represent a major hazard to organisms with lungs such as mammals including humans. The spread of airborne disease is rapid in enclosed spaces such as animal cages or pens, transport systems such as aircrafts and trains, prisons, public meeting places such as discos, schools and hospitals.

Farm animals and the general public have little or no protection against airborne disease which is one reason why airborne disease is reported to have created one of the greatest natural disasters that humankind has experienced.

### SUMMARY OF THE INVENTION

An object of the present invention is provide a system and method for the detection of pathologic states in mammals, especially respiratory diseases. This object is solved by methods, systems, devices and a computer program product as defined in the attached claims.

In particular, the present invention provides a computer based method for monitoring, e.g. the recognition of health, physical states or arousal or respiratory status of a mammal, comprising:

capturing a remote cough event using one or more sensors such as microphones,

analyzing the cough event to determine if it is indicative of a sick or healthy cough, and localizing the cough event.

The present invention provides a computer based system for the monitoring, e.g. recognition of health, physical states or arousal or respiratory status of a mammal, comprising:

one or more sensors such as microphones for capturing a remote cough event,

means for analyzing the cough event to determine if it is indicative of a sick or healthy cough, and

Means for localizing the cough event.

The present invention provides a portable electronic device having a processing engine and a memory, comprising:

one or more sensors such as microphones for capturing a remote cough event,

means for analyzing the cough event to determine if it is indicative of a sick or healthy cough, and

Means for localizing the cough event.

The means for analyzing may be adapted for real time operation and/or to use

Hidden Markov models or Dynamic Time warping.

The means for analyzing may optionally be adapted to use a first model that calculates characteristic parameter of the respiratory status from sound captured by the one or more microphones. The characteristic parameter may be one of spectral content, an autoregressive model parameter or acoustic energy.

The means for analyzing may also be adapted to use a second model to quantify the dynamic variation of the characteristic parameter. In addition the device may include means for classification of the cough event based on dynamic variation of the characteristic parameter.

The device may also comprise means for extraction of sound information from the sound signal captured by the one or more microphones, the means for extracting having:

means for calculating the energy of the sound signal,
means for calculating the Hilbert transform of the energy,
means for calculating the square root of the sum of the energy and its Hilbert transform, and
means for calculating the moving average of the result to get a smoothed estimate of the envelope of the initial signal.

Preferably the means for localizing the cough event comprises: means for estimation of a time difference of arrival of the sound signal captured by the one or more microphones.

Alternatively the means for localizing the cough event may comprise any of:

means for energy thresholding, and
means for detecting simultaneous movements of the mammal. The means for detecting simultaneous movements of the mammal include means for analysing images from a camera or means for comparing the sound signal captured by the one or more microphones with an output of a movement detector. The movement detector may be an accelerometer.

The present invention also provides a computer program product including code segments that when executed on a computing system implement any of the methods or devices of the present invention. The present invention also includes a machine readable storage medium storing the computer program product.

Specific individual embodiments of the present invention are defined in the attached claims and explained in more detail below.

### BRIEF DESCRIPTION OF THE DRAWINGS

FIGS. 1a and b show a human application of monitoring cough with a mobile phone or PDA in accordance with an embodiment of the present invention. Data can be sent wirelessly to a server, where spread of cough events and statistics can be visualized. FIG. 1c shows how a cough may be localized to a person carrying a portable device or remote therefrom in accordance with an embodiment of the present invention.

FIG. 2 shows an flow diagram according to an embodiment of the present invention.

FIG. 3 shows a sound extraction procedure. The cough sound (top plot), its energy (middle plot), the envelope of the energy (bottom plot) and the chosen threshold (horizontal line on the bottom plot).

FIG. 4 shows a continuous recording and the extracted sounds are shown.

FIG. 5 presents the center and the boundaries of the cluster on the (a1, a3) plane. 88% of the sick cough are correctly identified, achieving a 92% of correct overall classification rate.

FIG. 6 shows the trace of a triangle sound received in 2 of the microphones.

FIG. 7 shows a three dimensional graph for the weight w(k, l) for every position (k, l).

FIG. 8 shows an output of cough localization algorithm according to an embodiment of the present invention.

FIG. 9 shows an output of an image analysis algorithm that can be used simultaneously with an acoustic cough monitoring system according to any embodiment of the present invention.

FIG. 10 shows a computing system schematically such as in a mobile phone, PDA, laptop or personal computer for use with the present invention.

FIG. 11 shows a scheme for monitoring and labelling bioresponses according to an embodiment of the present invention.

### DESCRIPTION OF THE ILLUSTRATIVE EMBODIMENTS

The present invention will be described with respect to particular embodiments and with reference to certain drawings but the invention is not limited thereto but only by the claims. The drawings described are only schematic and are non-limiting. In the drawings, the size of some of the elements may be exaggerated and not drawn on scale for illustrative purposes.

Furthermore, the terms first, second, third and the like in the description and in the claims, are used for distinguishing between similar elements and not necessarily for describing a sequential or chronological order. It is to be understood that the terms so used are interchangeable under appropriate circumstances and that the embodiments of the invention described herein are capable of operation in other sequences than described or illustrated herein.

Moreover, the terms top, bottom, over, under and the like in the description and the claims are used for descriptive purposes and not necessarily for describing relative positions. It is to be understood that the terms so used are interchangeable under appropriate circumstances and that the embodiments of the invention described herein are capable of operation in other orientations than described or illustrated herein.

It is to be noticed that the term “comprising”, used in the claims, should not be interpreted as being restricted to the means listed thereafter; it does not exclude other elements or steps. Thus, the scope of the expression “a device comprising means A and B” should not be limited to devices consisting only of components A and B. It means that with respect to the present invention, the only relevant components of the device are A and B.

Similarly, it is to be noticed that the term “coupled”, also used in the claims, should not be interpreted as being restricted to direct connections only. Thus, the scope of the expression “a device A coupled to a device B” should not be limited to devices or systems wherein an output of device A is directly connected to an input of device B. It means that there exists a path between an output of A and an input of B which may be a path including other devices or means.

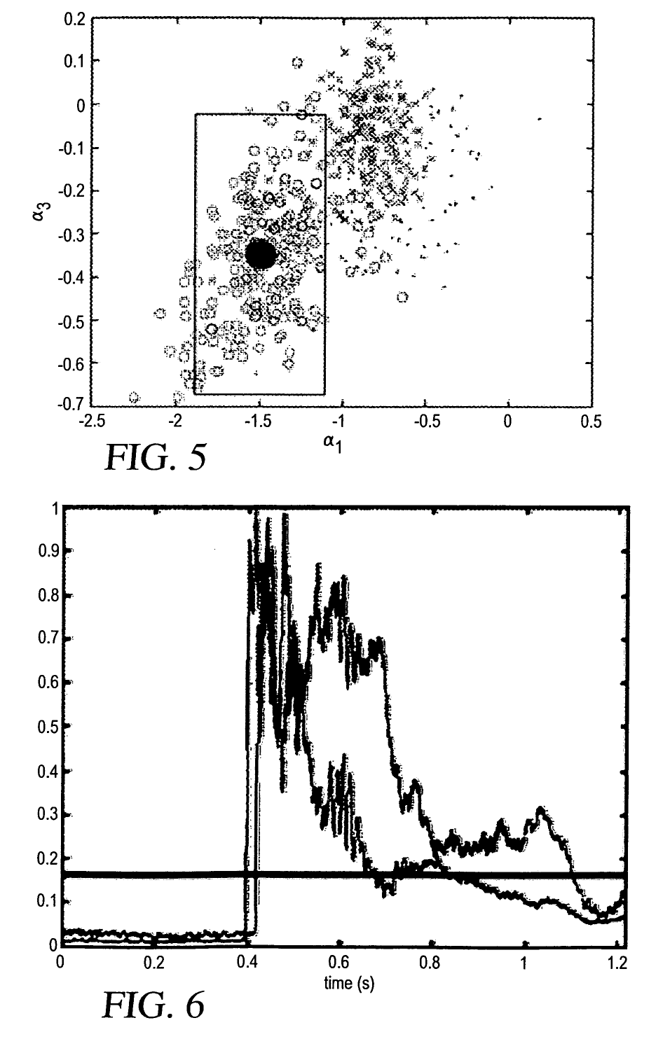

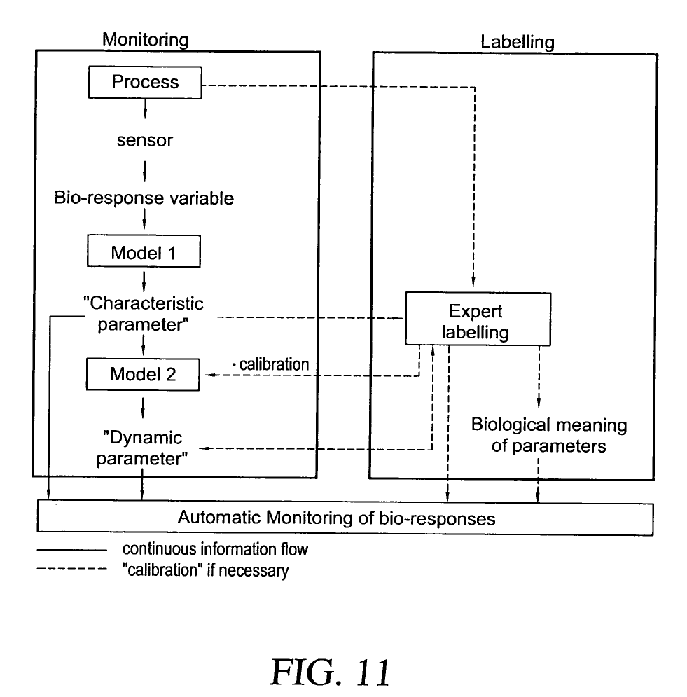

Referring to FIG. 11, the present invention proposes in one aspect a system and method for combining the respiratory status (e.g. amount and type of cough) with the localization of organisms having the respiratory status in real time. The organisms are able to suffer from a respiratory complaint, i.e. they have lungs such as mammals especially farm animals and humans. In particular the present invention is advantageous for animals and humans who are exposed to closed confinements such as pens, cages, aircraft, public places where humans are in close proximity to each other.
1. A so called bioresponse for example a “cough event” is measured continuously on one or more living organism(s) especially mammals including humans. A cough event is a physiological process in which a cough is produced or a sequence of coughs are produced (cough sequence). The cough can be non-spontaneous as well as spontaneous. The term non-spontaneous coughing, also denoted with intended or elicited coughing, refers to coughing which does not appear due to a pathological process as in the case for spontaneous coughing, but is directly forced. In other words non-spontaneous coughing is preceded by a particular intervention, e.g. nebulisation of an irritating substance in case of animal subjects or a request in case of human subjects. Examples of spontaneous coughs are human acute coughs or animal chronic coughs.
2. For the acquisition a microphone, several microphones or the combination of a microphone(s) with other sensors (like EMG, accelerometer, . . . ), a camera, or the combination of camera and microphone(s), or other sensors available for monitoring cough events are used.
3. Automatic cough identification is done in real-time, or semi-real time in which fragments of data are recorded and processed in segments.
4. Identification (or classification) of the cough event is done by means of any known sound classification algorithm (as Hidden Markov Models, Dynamic Time Warping, LPC, . . . ) but can also be model based. This means that identification is done based on the dynamic variation of the acquired signal in time. In this case a first model, model 1, is made from the measured bioresponse variable to calculate a relevant parameter, the so called “characteristic parameter”, for example “posture parameters” from an image or a “sound characteristic parameter” (like frequency content, ar-parameter) from a sound. This characteristic parameter is a model parameter from model 1 and is varying with the variable behavior or status of the respiratory system of the living organism. Consequently these characteristic parameters such as sound characteristic parameters or image characteristic parameters are varying as a function of time and their value is known a priori or by continuous updating the model. Continuous means that the sampling rate of the measurement is fast enough to measure all relevant responses of the living organism in relation to the considered variable. A second model (model 2) is made to quantify the dynamic variation of the characteristic parameters. The parameters of this second model, the so called “dynamic parameters”, are a measure for the dynamic variation of the characteristic parameter or their combination. These dynamic parameters will allow classification of the cough events. Observers might also quantify (by labeling) the limits or threshold for which the values of the dynamic parameters allow classification of cough events (see FIG. 5).
5. Identification might comprise the use of different sensors. Using the model based approach will allow the use of input-output models.
6. The method and system allows using models for individual monitoring (defined by the model structure and parameters) for better performance (classification and localization).
7. The method and system has a localization of the cough event. This can be done by using multiple microphones. Localisation means in this context that a cough event is associated with a possible location of an organism from which the cough originates, the location being remote from one or more of the microphones. This means that the organism whose cough event is captured by the microphones is not carrying one of the microphones that are used. This has the advantage that organisms such as farm animals, e.g. pigs or humans do not need to carry a transponder having a microphone. This saves cost and allows detection of third party animals whose respiratory disease may be of danger to others. The localisation may be obtained, for example, from the time delay of arrival from a cough sound to the different microphones. This time delay can be measured or can be calculated and used to triangulate the region from which the cough originates.

Based on the dimensions of the monitored environment a special representation can be provided showing the location and amount and/or type of cough(s). The method can comprise the use of other localization systems (like GPS, Galileo) for transmitting the amount/type of coughs detected at a specific location.
8. The method and system comprise an alert system, allowing immediate feedback of the cough monitor to the user. This can be done by means of any suitable telecommunications method of which SMS (Short Message Service), MMS, email or other information services are only examples. For example, a farmer or vet can receive an SMS with the number (and/or type) of coughs detected, the infected pens, the spread rate of the coughs, etc. Example on humans: person using a cough identification algorithm gets informed about the number and types of registered coughs.
9. The information can be put on a server used by a variety of users to visualize the occurrence of coughs on animal cage or pen level, compartment level, farm level, province level, country level, or global level. This can be accessible via internet or a shared server. This also can be useful to see the spread of respiratory disease of humans.
10 The method and system allows optimized management towards the use of medication. The user can take action to inform a vet, or in case the vet is informed himself he can take the necessary steps towards medication. This will allow smaller scale treatment of organisms, for example only injecting the infected pens and not the entire stable which might reduce the use of antibiotics. Another application is the adjustment of medication in humans using the dynamic response of the respiratory system (occurrence of cough in time) on previous medication or environment.
11. The method and system allows prediction of the evolution of cough events (number or type) in the future. This can be used for feedback on medication.
12. The method and system can comprise the use of environmental sensors or local environmental data including temperature, humidity and contaminant concentrations. Mapping the occurrence of cough events with environmental data might give insight in cause of health distress.
13 The method and system can provide information for adjustment of medication in humans using the response of the subject (like allergy) to environmental variables. This could also be coupled to an agenda for monitoring the response in time.
14 The method and system can also be used for sneezing. By combining with environmental data (wearable sensor or satellite observations) the cause of allergies might be unveiled.

### Example on Human Coughs:

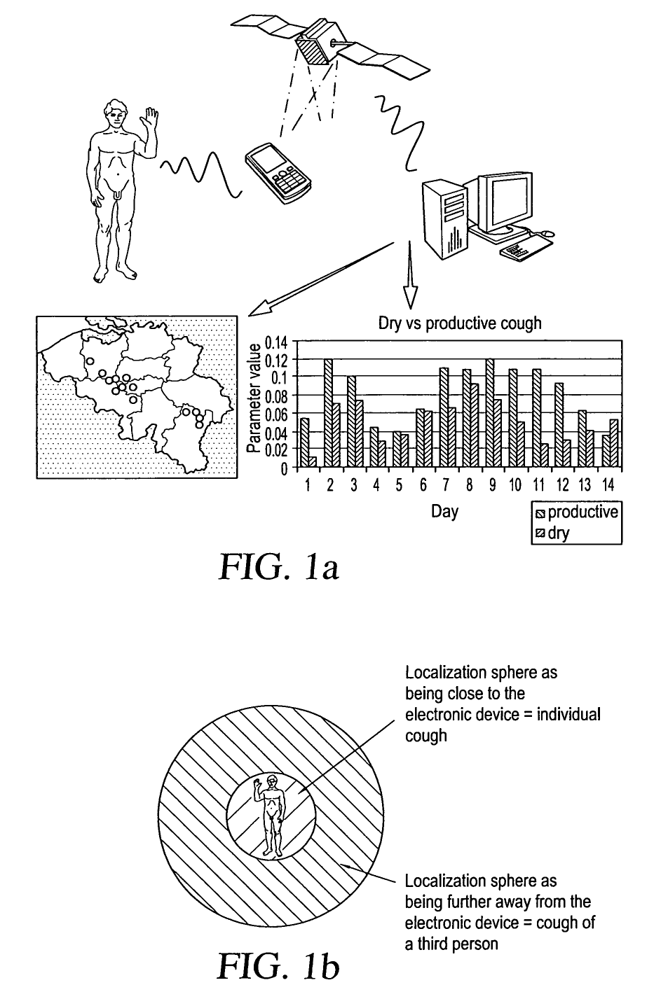

A cough detection algorithm is implemented on a mobile phone or PDA or laptop or other portable electronic device processing in real time the occurrence of the number of coughs and/or the type of coughs. This information is stored on the electronic device. The information can be stored together with the location of the cough. Optionally the location and the information can be transmitted to a server storing all such information (e.g. ID number, amounts of cough, type of cough, location). For example, using a password, a user can have access to the data, e.g. showing the number of coughs in time in a graph, together with the location of coughing. Governments and national or private health agencies can use this information from several users to gain information about the spread of respiratory disease (FIG. 1a). Cough events can be quantified from users, useful for diagnostic or health management reasons.

In a particular embodiment of the present invention a cough detection algorithm is used to determine, e.g. by localization of coughs, whether the cough belongs to the carrier of the portable device (e.g. mobile phone, PDA, laptop) or to a third party—see FIG. 1b. Hence the portable device is used for remote sensing of coughs of third parties as well as localization of these coughs, i.e. that they are not from the person carrying the portable device but from a remote person. Localisation also means in this embodiment that a cough event is associated with a possible location of a human from which the cough originates, the location being remote from the microphone in the portable device. This means that the third party human whose cough event is captured by the microphones is not carrying one of the microphones that are used. This has the advantage that the humans being examined do not need to carry a transponder having a microphone. The localization of the cough to or not to the person carrying the portable device can be made by means of, for example, energy thresholding, e.g. to determine if the energy threshold is too low so that it must come from a remote third party. Alternatively, simultaneous movements can be recorded, e.g. from a means for detecting movement such as from the output of an accelerometer already built in the electronic device. If the cough comes from the person wearing or carrying the portable device this device will usually be subject to a movement that can be detected by the means for detecting movement such as an accelerometer. If the accelerometer gives no output at the same time as a cough event is detected via the microphone or microphones, the cough probably comes from a third party, i.e. the cough is localized as a remote cough (see FIG. 1c). Alternatively, a second electronic device containing an accelerometer can be used which communicates with the portable device running the cough algorithm device. By detecting a cough as belonging to the carrier of the cough algorithm, the method can differentiate coughs from third parties located at a certain distance. The use of algorithms for exclusion of background sound noise may also provide a method for locating coughs originating from the carrier of the device and a third person.

### Example Pigs Cough Monitoring Using Microphones

1. At least one sensor (and/or microphone) is used for data acquisition, of which acoustic characteristics of sound from the animals is calculated (model 1). This first model will estimate or calculate the required parameters for cough recognition. Some characteristic parameters are spectral content, autoregressive model parameters or the shape of the acoustic energy contained in the signal.

2. These characteristic parameters will be calculated or estimated per time window. A sequence of these parameters will give a time series of characteristic parameters.

3. A second model (model 2) is made in which dynamic features of the time series of characteristic parameters are estimated. These dynamics of the features of a bioresponse (like a cough event) will be described by the dynamic model parameters, which will allow classification of the bioresponse (cough events). The performance of the classifier is guaranteed via labelling in which an updated discrimination method is provided when necessary. Several events of coughing might be registered in time. When more microphones are used, the position of the cough event is derived. This can be done by using the time delay of arrival between the microphones, or other techniques in which positioning is possible. This results in a map which shows the 2D distribution of cough events (FIG. 5). The model used for cough classification might also discriminate between types of cough, like healthy or sick. This information can inform farmer or vets where sick animals are located, so selective treatment is possible. Such an application could lead to a decrease use of medication like antibiotics, or can serve as an early warning system on which the farmer or vet can respond by direct contact (feed or medication) or by changing the environment. The method described in this example can be adapted individually, for example when applied in different stables. By measuring the building characteristics, the model for classification can be adjusted (calibration of the system). A similar technique can be used for human cough detection by using microphone(s) and/or, accelerometer(s) (or other sensor) or a combination sensors.

### Signal Analysis

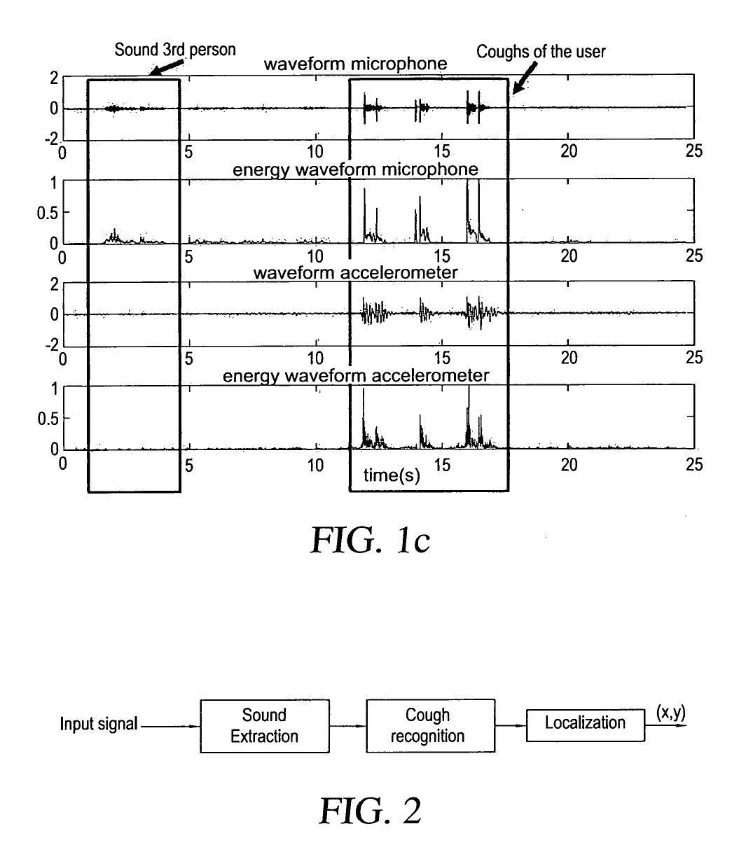

The flow chart for the proposed application for cough recognition and localization is shown if FIG. 2 and comprises mainly of three subprocesses, namely the sound extraction from the sound signal received from one or more microphones, the cough recognition and the localization, that are presented in the following in more detail.

### Sound Extraction

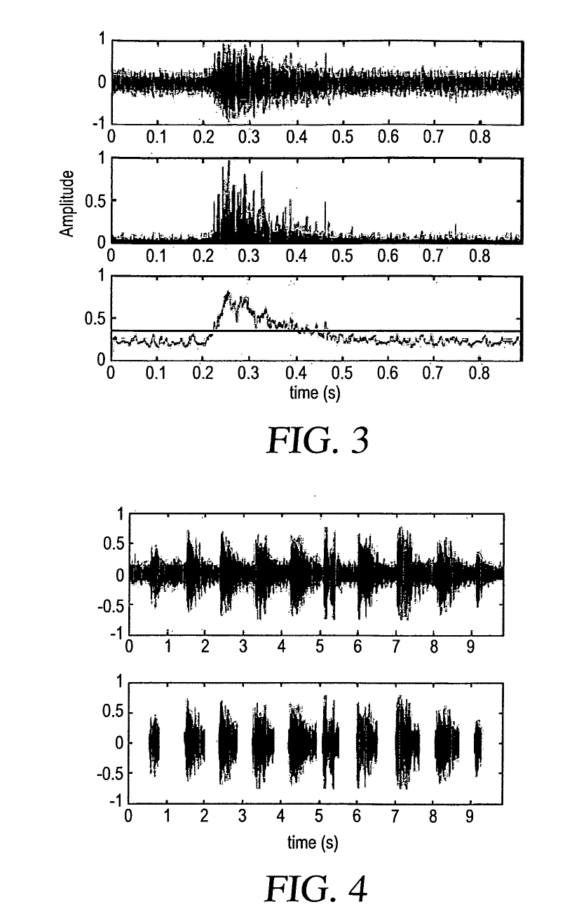

The extraction of individual sounds from a continuous recording is based on the envelope of the energy of the signal and a selected (environment specific) threshold as is presented in FIG. 3.

The underlying principle is that low amplitude noise is recorded most of the time and when a sound occurs (any sound within the pig farm) will be recorded as a high energy signal. Whenever the amplitude of the envelope is higher than the threshold it is considered that there is a recording of a sound that needs to be identified. The mean value of the envelope over the complete recording is used for this application and experimentation suggested that it is adequate for extracting most of the signals that are of interest.

The Hilbert transform of a discrete time signal s[k] that is defined as:

$$ℋ \{ s [ k ] \} = {\sum }_{n = - N / 2}^{N / 2} s [ k - n ] h [ n ] {sin}^{2} ( \frac{n π}{2} )$$

where

$$h [ k ] = \frac{2}{k * π} ,$$

for

$$k = \pm 1 , \pm 2 , … , \pm \frac{N}{2}$$

and h[0]=0, and provides a 90° phase shift to the original signals use to automatically extract the envelope according to the following steps:
1. Calculate the energy of the signal
2. Calculate the Hilbert transform of the energy
3. Calculate the square root of the sum of the energy and its Hilbert transform
4. Calculate the moving average of the result to get a smoothed estimate of the envelope of the initial signal

The result of this procedure is presented in FIG. 4, where a continuous recording is presented and the extracted sounds are shown.

It is suggested that the mean value of the signal is adequate for the sound extraction procedure, but it should be noted that the noise level and the acoustics of the pig compartment affect the resulting signal, and the threshold should be chosen taking them into account.

### 2. Sick Cough Recognition

a. Preprocessing of Individual Sounds

The sounds that usually occur in a pig compartment are pig movements, pig vocalizations, metal sounds (e.g. clanging of compartment doors) and low frequency noise caused primarily by ventilation fans. After the extraction of individual sounds from a continuous recording, a preprocessing takes place. To make sure that the sound extraction is correct, sounds that are very close to each other are considered as one. To clarify this, consider the case of a scream. It consists of the inhalation phase in which the animal inhales air into its lungs and the exhalation in which the production of the sound occurs. However, multiple repetitions of these phases might create a single scream while the envelope extraction may result in different sounds for each repetition of inhalation and exhalation. By preprocessing, this effect is avoided and results in complete sound signals only. Experimentation suggested that sounds that are closer than 100 ms be considered as a single sound. Furthermore, the length of each sound contains information that can be used in classification. Screams and grunts for example, are longer sounds that can last for up to a few seconds. Coughs on the other hand are sharp sounds that usually last from 200 ms up to 600 ms4. Sounds longer than 600 ms are therefore considered as non-interesting and ignored from the rest of the process. Although it is unlikely for sounds shorter than 200 ms to occur, this case is also considered and short sounds are also eliminated.

b. Auto Regression Analysis and Cough Recognition

Autoregressive (AR) analysis, is a method of estimating a signal s[k] with one of the form:

$$\overline{s} [ k ] = - {\sum }_{i = 1}^{p} {a}_{i} s [ k - i ] + e [ k ] \qquad ( 2 )$$

where the αᵢ, i=1, 2, . . . , p are estimated minimizing an adequate criterion²², e[k] is a white noise signal and p is the order of the AR model

It has been shown that a connection can be made between Data Based Mechanistic (DBM) models and physical models. Although such a connection has not been made in this work, it is clear that such a connection exists and remains to be found. Based on this assumption and the study of the time domain characteristics of pig vocalizations an attempt to form a classifier is made. In this regard, it is observed that the positions of the AR parameters in a 3 dimensional space for the laboratory sounds of the first data set can serve as an adequate and computationally efficient classifier. It is suggested that plotting the AR parameters on the (a1, a2, a3) space they tend to form a cluster of sick pig cough sounds. FIG. 5 for example shows the (a1, a3) plane and the mapping of the different sounds.

To gain an insight of the classification properties of the proposed method, the center (c1, c2, c3) of the sick cough cluster is defined as the mean value of the AR parameters of 5 randomly selected pre-labeled sick coughs. Its boundaries are defined as the vertices of the polyhedron whose edges in any direction ai equals the twice of the standard deviation of the training set in that direction. The length of each edge is based on the Chebyshev inequality according to which at least 75% of the training set values will be within this area. FIG. 5 presents the center and the boundaries of the cluster on the (a1, a3) plane. 88% of the sick cough are correctly identified, achieving a 92% of correct overall classification rate.

Although it cannot be considered as a reliable measure since previous knowledge of the dataset can be considered (yet the choice of the training set is random), it provides an indication as to the result that can be achieved.

### 3. Localization

a. Estimation of the Time Difference of Arrival (TDOA)

The localization algorithm presented in section II.B.3.b requires the estimation of the TDOA of the signal on the 7 microphones in the pig house. Although many methods have been presented in the literature as described in the introduction, in this paper the envelope of the energy of the signal (FIG. 3) is used to estimate the time at which a sound starts. Then the difference of initiation times for each pair of microphones is the TDOA between them.

After the extraction of the envelope, the envelopes of the signals received in all microphones are normalized and their mean value is taken. The smallest of these values is used as a threshold to define the amplitude at which a sound is considered to begin. This is graphically presented in FIG. 6 for a triangle sound received in 2 of the microphones.

b. The Localization Algorithm

If a sound originates from a certain position, the difference in the capturing times of this signal in two microphones, due to their different distance from the sound, results in a time delay between two microphones. By multiplying this time delay by the travelling speed of sound (343.4 m/s at 20° C.), a distance can be calculated. Let us call this the distance of the time delay, d_. Let dpa be the distance from a certain point p in the pig house to microphone a, and dpb the distance between the same point and microphone b. If at this specific point p a sound signal would be released, it would travel the distance dpa to microphone a and distance dpb to microphone b. At this point p, the distance of the time delay d_is equal to the difference dpa−dpb, so in the weight wp for the position p is:

wp=(dpa−dpb)−d_{—}=0 (3)

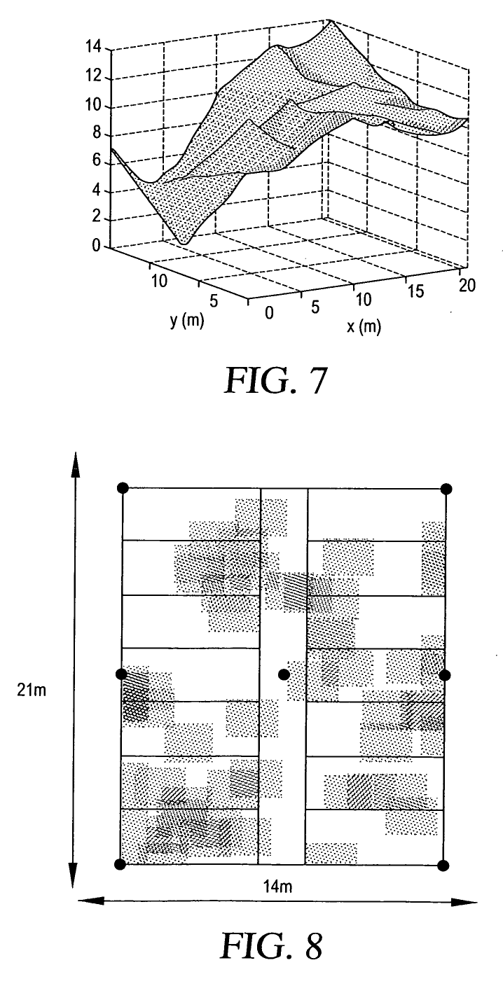

In order to find the potential location of a sound source, the positions at which w=0 should be found. To compute these weights w, the test field is divided in a 2 dimensional grid with a resolution of 0.1 m. As the pig house is 21 m by 14 m, this implies a grid with a resolution of 210×140. In every point of the grid the weight 3 for every pair of microphones and summed. This is represented in the following equation:

$${w}_{( k , l )} = {\sum }_{i = 1}^{n - 1} {\sum }_{j = i + 1}^{n} \{ ( {d}_{( k , l ) , i} - {d}_{( k , l ) , j} ) - {d}_{τ ( i , j )} \} \qquad ( 4 )$$

where w(k, l) represents the total weight at position (k, l), (d_{(k,l),j}−d_{(k,l,),j}) the difference in distance between position (k,l) and microphones i and j the distance of the time delay between the signals at microphone i and j, and n the number of microphones, By calculating this weight w(k, l) for every position (k, l), the total area can be visualized in a three dimensional graph. An example of this graph is shown in FIG. 7.

The position of the sound source is that point in the grid where the weight w(k, l) is minimal, i.e. the position at which the minimum of the graph is located. It could be argued that knowledge of the pig house geometry and the TDOA between the microphones would simplify the problem to one finding the point at which all equations of the form (3) would be satisfied for every pair of microphones. However, this would require a very accurate estimation of the TDOA leading to increased complexity of that algorithm. On the contrary, the algorithm presented above is more robust to TDOA uncertainty and therefore, the procedure that calculates it (section II.B.3.a) can be very simple. The overall result is shown in FIG. 8, where the 2D spread of cough events is shown.

To test the sensitivity of the proposed algorithm, the triangle sounds of the second data set are used. The real (measured values) and estimated values of the sound positions are presented in Table I.

TABLE 1
Results of the triangle sounds experiments for the evaluation of the
sensitivity of the localization algorithm

| | X coordinate (m) | | Y coordinate (m) | |
|---|---|---|---|---|
| Test | Measured/Estimated | Error | Measured/Estimated | Error |
| 1 | 10.8/10.0 | 0.8 | 1.3/1.9 | 0.6 |
| 2 | 10.8/10.8 | 0.0 | 3.9/3.9 | 0.0 |
| 3 | 10.8/12.1 | 1.3 | 6.5/5.9 | 0.6 |
| 4 | 10.8/11.0 | 0.2 | 9.2/9.0 | 0.2 |
| 5 | 10.8/11.3 | 0.5 | 11.8/11.9 | 0.1 |
| 6 | 10.8/11.4 | 0.6 | 14.4/15.0 | 0.6 |
| 7 | 10.8/10.7 | 0.1 | 17.0/17.0 | 0.0 |
| 8 | 10.8/11.2 | 0.4 | 19.7/19.5 | 0.2 |
| 9 | 3.2/0.1 | 3.1 | 1.3/0.1 | 1.2 |
| 10 | 3.2/3.1 | 0.1 | 19.7/19.2 | 0.5 |
| 11 | 3.2/2.8 | 0.4 | 17.0/16.6 | 0.4 |
| 12 | 3.2/3.0 | 0.2 | 14.4/14.6 | 0.2 |
| 13 | 3.2/2.7 | 0.5 | 11.8/11.7 | 0.1 |
| 14 | 3.2/0.1 | 3.1 | 9.2/0.1 | 9.1 |
| 15 | 3.2/2.7 | 0.5 | 6.5/6.3 | 0.2 |
| 16 | 3.2/2.7 | 0.5 | 3.9/4.1 | 0.2 |

It is noted that the positioning of tests 9 and 14 do not give the desired results. By examining the time domain signals in these cases, it is observed that the algorithm fails to correctly estimate the TDOA. The extracted signals don't correspond to the actual sounds due to high surrounding noise, which results to algorithm failure. However, the overall results suggest that the algorithm is of adequate accuracy for this specific application.

Example Cough Recognition with Camera's

The sensor in accordance with this embodiment is a top-view camera collecting images, in which the change of an individual subject's position and body shape (=the bioresponse) during cough is visible, e.g. for an animal, especially a farm animal such as a pig or for a human.

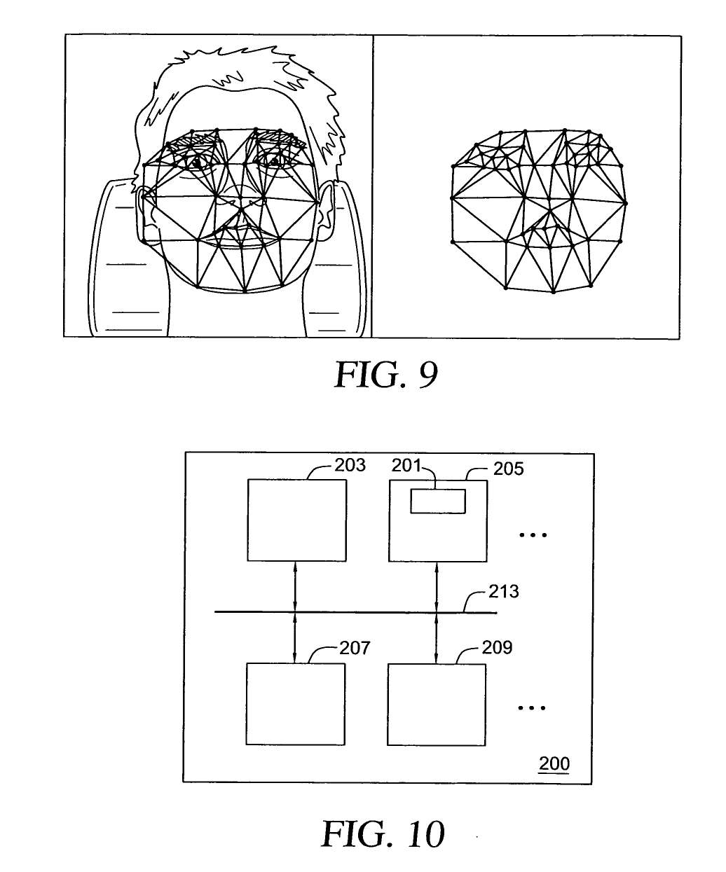

A model is made (model 1), describing the subject's position, posture or body shape, e.g. for an animal, especially a farm animal such as a pig or for a human. The model is for example a mathematical ellipse shape where position, orientation or shape (length and width) are defined by the model's posture parameters. The ellipse can be extended to a 32 point (or more) body contour model fitted through the image of a mammal or for example a model connecting 52 points on a human face (see FIG. 9). This method can also be used on demented elderly. This model is updated to fit the contour of the subject (e.g. for an animal, especially a farm animal such as a pig or for a human) in all subsequent images from the camera, resulting in a time series of posture parameters. The posture parameters are kept as continuous values and not classified as discrete posture classes so that they contain the individual nature of the individual dynamic subject's bioresponse (e.g. of an animal, especially a farm animal such as a pig or of a human).

A second model is made (model 2) describing the variation of the posture parameters as a function of time, corresponding to the behavior of the subject, e.g. of an animal, especially a farm animal such as a pig or of a human. The so called dynamic model parameters of the second model are also updated continuously to account for the changing behavior of the subject, e.g. of an animal, especially a farm animal such as a pig or of a human.

Coughs of an individual subject (e.g. of an animal, especially a farm animal such as a pig or of a human) can be classified from the image measurements when the dynamic model parameters fall within limits which are defined by labeling.

### Implementation

Embodiments of the present invention can comprise control software in the form of a computer program product which provides the desired functionality when executed on a computing device, e.g. a laptop, a personal computer, a mobile phone, a PDA. Further, the present invention includes a data carrier such as a CD-ROM or a diskette which stores the computer product in a machine readable form and which executes at least one of the methods of the invention when executed on a computing device. Nowadays, such software is often offered on the Internet or a company Intranet for download, hence the present invention includes transmitting the computer product according to the present invention over a local or wide area network. The computing device may include one of a microprocessor and an FPGA.

The above-described method embodiments of the present invention may be implemented in a processing system 200 such as shown in FIG. 10. FIG. 10 shows one configuration of processing system 200 that can be implemented on a mobile phone, a PDA, a laptop, a personal computer etc. It includes at least one programmable processor 203 coupled to a memory subsystem 205 that includes at least one form of memory, e.g., RAM, ROM, and so forth. It is to be noted that the processor 203 or processors may be a general purpose, or a special purpose processor, and may be for inclusion in a device, e.g., a chip that has other components that perform other functions. The processor may also be an FPGA or other programmable logic device. Thus, one or more aspects of the present invention can be implemented in digital electronic circuitry, or in computer hardware, firmware, software, or in combinations of them. The processing system may include a storage subsystem 207 that has at least one disk drive and/or CD-ROM drive and/or DVD drive. In some implementations, a display system, a keyboard, and a pointing device may be included as part of a user interface subsystem 209 to provide for a user to manually input information. Ports for inputting and outputting data also may be included, especially interfaces for one or more microphones for capturing sound signals from organisms such as mammals, especially for capturing cough events. Further interfaces may be provided for coupling image capturing devices to the computer system, e.g. for connection to a digital camera or cameras, e.g. a video camera. More elements such as network connections, interfaces to various devices, and so forth, may be included, either by wireline or wireless connections, but are not illustrated in FIG. 10. The various elements of the processing system 200 may be coupled in various ways, including via a bus subsystem 213 shown in FIG. 10 for simplicity as a single bus, but will be understood to those in the art to include a system of at least one bus. The memory of the memory subsystem 205 may at some time hold part or all (in either case shown as 201) of a set of instructions that when executed on the processing system 200 implement the steps of the method embodiments described herein. Thus, while a processing system 200 such as shown in FIG. 10 is prior art, a system that includes the instructions to implement aspects of the methods for characterising a sample fluid is not prior art, and therefore FIG. 10 is not labelled as prior art.

The present invention also includes a computer program product, which provides the functionality of any of the methods according to the present invention when executed on a computing device. Such computer program product can be tangibly embodied in a carrier medium carrying machine-readable code for execution by a programmable processor. The present invention thus relates to a carrier medium carrying a computer program product that, when executed on computing means, provides instructions for executing any of the methods as described above. The term “carrier medium” refers to any medium that participates in providing instructions to a processor for execution. Such a medium may take many forms, including but not limited to, non-volatile media, and transmission media. Non volatile media includes, for example, optical or magnetic disks, such as a storage device which is part of mass storage. Common forms of computer readable media include, a CD-ROM, a DVD, a flexible disk or floppy disk, a tape, a memory chip or cartridge or any other medium from which a computer can read. Various forms of computer readable media may be involved in carrying one or more sequences of one or more instructions to a processor for execution. The computer program product can also be transmitted via a carrier wave in a network, such as a LAN, a WAN or the Internet. Transmission media can take the form of acoustic or light waves, such as those generated during radio wave and infrared data communications. Transmission media include coaxial cables, copper wire and fibre optics, including the wires that comprise a bus within a computer.

1. A computer based method for monitoring a mammal, comprising:
capturing a remote cough event using one or more sensors,
analyzing the cough event to determine if it is indicative of a sick or healthy cough, and
localizing the cough event.

2. The method of claim 1, wherein the one or more sensors are one or more microphones

3. The method of claim 1, wherein the analyzing is done in real time.

4. The method of claim 1 wherein the analyzing includes any of Hidden Markov models or Dynamic Time warping, LPC, ARX-models or input-output models.

5. The method of claim 1, wherein the analyzing includes a first model that calculates characteristic parameter of the respiratory status from sound captured by the one or more microphones.

6. The method of claim 5, wherein the characteristic parameter is one of spectral content, an autoregressive model parameter or acoustic energy.

7. The method of claim 5, wherein the analyzing includes a second model to quantify the dynamic variation of the characteristic parameter.

8. The method of claim 7, further comprising classification of the cough event based on dynamic variation of the characteristic parameter.

9. The method of claim 1 further comprising extraction of sound information from the sound signal captured by the one or more sensors, by:
Calculating the energy of the sound signal,
Calculating the Hilbert transform of the energy,
Calculating the square root of the sum of the energy and its Hilbert transform,
Calculating the moving average of the result to get a smoothed estimate of the envelope of the initial signal.

10. The method of claim 1 wherein localizing the cough event comprises:
estimation of a time difference of arrival of the sound signal captured by the one or more microphones.

11. The method of claim 1, wherein localizing the cough event comprises any of:
energy thresholding, and
detecting simultaneous movements of the mammal.

12. The method of claim 11, wherein detecting simultaneous movements of the mammal is carried out by means of analysing images from a camera or by comparing the sound signal captured by the one or more microphones with an output of a movement detector.

13. The method of claim 12, wherein the movement detector is an accelerometer.

14. A computer based system for the recognition of respiratory status of a mammal, comprising:
one or more sensors for capturing a remote cough event,
means for analyzing the cough event to determine if it is indicative of a sick or healthy cough, with in both cases the possibility to classify it as a stress sick cough or a normal cough and
Means for localizing the cough event.

15. The system of claim 14, wherein the means for analyzing is adapted for real time operation.

16. The system of claim 14, wherein the means for analyzing is adapted to use Hidden Markov models or Dynamic Time warping, LPC, ARX models or input-output models.

17. The system of claim 14, wherein the means for analyzing is adapted to use a first model that calculates characteristic parameter of the respiratory status from sound captured by the one or more microphones.

18. The system of claim 17, wherein the characteristic parameter is one of spectral content, an autoregressive model parameter or acoustic energy.

19. The system of claim 17, wherein the means for analyzing is adapted to use a second model to quantify the dynamic variation of the characteristic parameter.

20. The system of claim 19, further comprising means for classification of the cough event based on dynamic variation of the characteristic parameter.

21. The system of claim 14 further comprising means for extraction of sound information from the sound signal captured by the one or more microphones, the means for extracting having:
means for calculating the energy of the sound signal,
means for calculating the Hilbert transform of the energy,
means for calculating the square root of the sum of the energy and its Hilbert transform, and
means for calculating the moving average of the result to get a smoothed estimate of the envelope of the initial signal.

22. The system of claim 14 wherein the means for localizing the cough event comprises: means for estimation of a time difference of arrival of the sound signal captured by the one or more microphones.

23. The system of claim 14, wherein the means for localizing the cough event comprises any of:
means for energy thresholding, and
means for detecting simultaneous movements of the mammal.

24. The system of claim 23, wherein the means for detecting simultaneous movements of the mammal has means for analysing images from a camera or means for comparing the sound signal captured by the one or more microphones with an output of a movement detector.

25. The system of claim 24, wherein the movement detector is an accelerometer.

26. The system of claim 14 in which relevant information can be combined with environmental data selected from temperature, dust, pollutants and humidity.

27. A portable electronic device having a processing engine and a memory, comprising:
one or more microphones for capturing a remote cough event,
means for analyzing the cough event to determine if it is indicative of a sick or healthy cough, and
Means for localizing the cough event.
